(19) United States
(12) Patent Application Publication
(10) Pub. No.: US 2010/0028935 A1
(43) Pub. Date: Feb. 4, 2010

(54) CONVEX BOTTOM MICROWELL

(76) Inventors: Carlo Ciaiolo, Airasca (IT); Marcella Rebola, Airasca (IT); Silvano Battaglio, Torino (IT); Alessandro Valerio, Airasca (IT); Claudio Santoro, Torino (IT); Annalisa Ciocchetti, Novara (IT); Umberto Dianzani, Torino (IT); Emanuele Albano, Torino (IT)

Correspondence Address: NIXON & VANDERHYE, PC, 901 NORTH GLEBE ROAD, 11TH FLOOR, ARLINGTON, VA, 22203, US

(21) Appl. No.: 12/515,129

(22) PCT Filed: Nov. 19, 2007
(86) PCT No.: PCT/IB07/03624

§ 371 Date: May 15, 2009

(30) Foreign Application Priority Data
Nov. 23, 2006 (IT) T02006A000833

(51) Int. Cl. C12Q 1/02; C12M 1/00; C12Q 1/00

(52) U.S. Cl. 435/29; 435/283.1; 435/4

(57) ABSTRACT

Convex-bottom microwells for laboratory use in optical analyses of colorimetric or enzymological type useful for avoiding interferences with the luminous beam of the detecting instrument by corpuscular elements present in the sample to be analysed.

The present invention concerns microwells for microbiological and/or enzymological analyses.

Diagnostic tests and microplate-based cell culture methods, that is, the study and growth of human cells or cells of animal origin, for longer or shorter periods of time, are now consolidated.

Tests of different nature can be performed directly in this culturing environment: from cell stimulation to drug resistance tests, from vitality reactions to metabolite dosage, and so on.

Microbiological and enzymological analyses are normally preformed in microplates that generally foresee the use of a detection system based on the development of a calorimetric reaction. That is, it is possible to perform colorimetric, immunocolorimetric, enzymatic tests, etc.

Therefore, in order to detect and quantify the calorimetric reaction, it is indispensable to reduce as much as possible the interference of the sample analysed with the luminous beam of the detecting instrument, generally a spectrophotometric microplate reader, where the interfering components are essentially constituted by the corpuscular elements present in the sample, which present an obstacle for the path of the luminous beam, impeding detection.

Therefore, particular attention must be paid to the cellularity of the culture in the case of microbiological analysis and, in particular, to the number and dimensions of the cells, which, if excessive, could constitute and obstacle to the path of the luminous beam. In addition, if the cells in culture derive directly from a haematic sample, it is important to remove all of the other corpuscular elements, such as blood cells and platelets.

The microplates currently used to perform enzymological and/or microbiological analysis are constituted by flat or concave bottomed microwells. The microplates in common use are indicated as flat-bottom, U-bottom or V-bottom and are chosen by the operators based on the different types of analyses to be preformed: cell culture, ELISA, etc.

The present invention has the scope of providing microwells configured so as to reduce events interfering with the detection operation performed with a detecting instrument.

Such scope is achieved thanks to the solution referred to specifically in the claims that follow. The claims form an integral part of the technical teaching provided here relative to the invention.

In a particular embodiment, such scope is achieved through microwells including a protuberance on the bottom of the microwell so as to create a peripheral collecting chamber.

In fact, the configuration of the above said microwells, called convex bottom, allows the concentration in the peripheral chamber on the bottom of the microwell of the corpuscular elements present in the liquid phase to be analysed, for example by centrifugation. In this way, it is possible to perform tests directly on whole blood or in conditions of critical cellularity and cellular dimension (for example, leukaemic blasts) without the occurrence of interferences such to invalidate the result of the qualitative or quantitative operation of the determination of the detection of the reaction.

In addition, a convex-bottom microwell can be employed advantageously to perform diagnostic analyses that have recourse to colorimetric reactions, such as, for example, the ELISA tests, when the solid phase is, for example, constituted by a plurality of beads on which it is possible to immobilise an analysis reagent, such as the capturing reagent. In fact, the presence of such beads dispersed in the microwell represents an element of interference with the operation of detection of the calorimetric reaction.

Furthermore, the present invention is directed to microplates having a plurality of convex-bottom microwells (for example, 4, 6, 12, 24, 96, 384), as well as to the use of such microwells of such microplates for performing microbiological and enzymological analysis on blood, plasma, serum or cell culture samples.

The present invention will now be described in detail as a non-limiting example with reference to the attached figures in which:

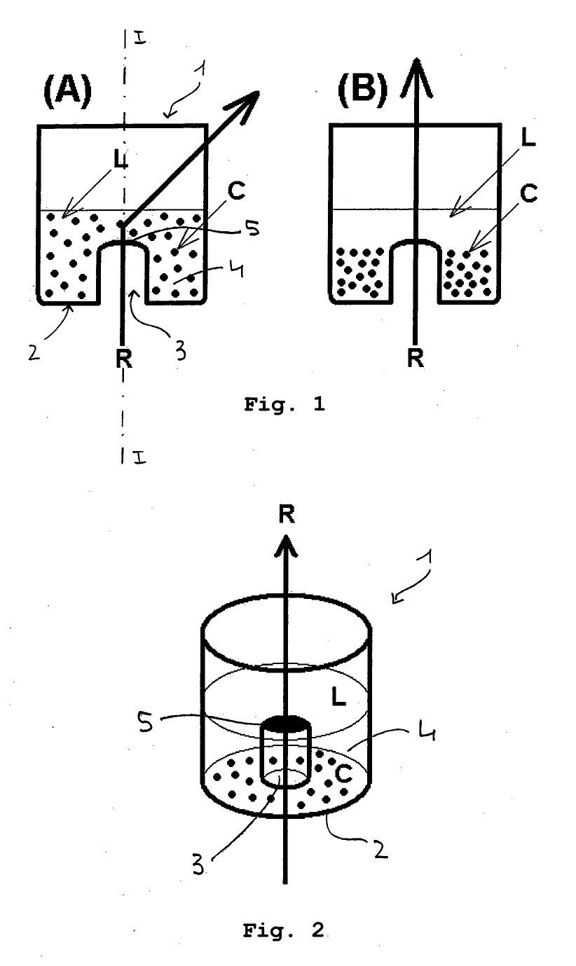

FIGS. 1A and 1B represent—in section—an embodiment, according to the present invention, of a convex-bottom microwell containing a corpuscular sample before (A) and after (B) centrifugation;

FIG. 2 represents—in prospective view—an embodiment of a microwell according to the present invention;

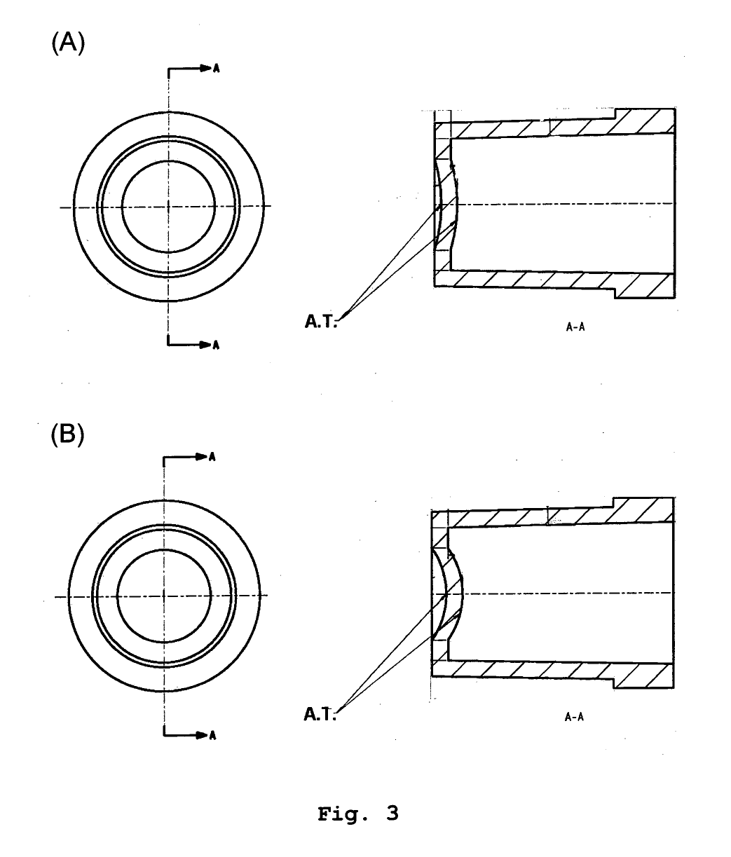

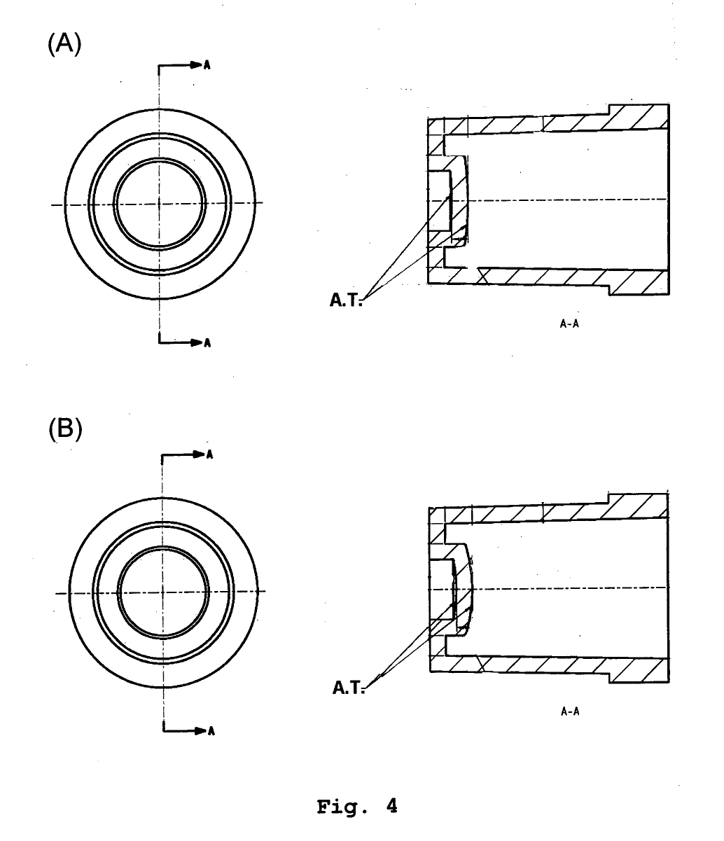

FIGS. 3A, 3B, 4A and 4B reproduce the views in plan and in section, of four different embodiments of a microwell according to the present invention;

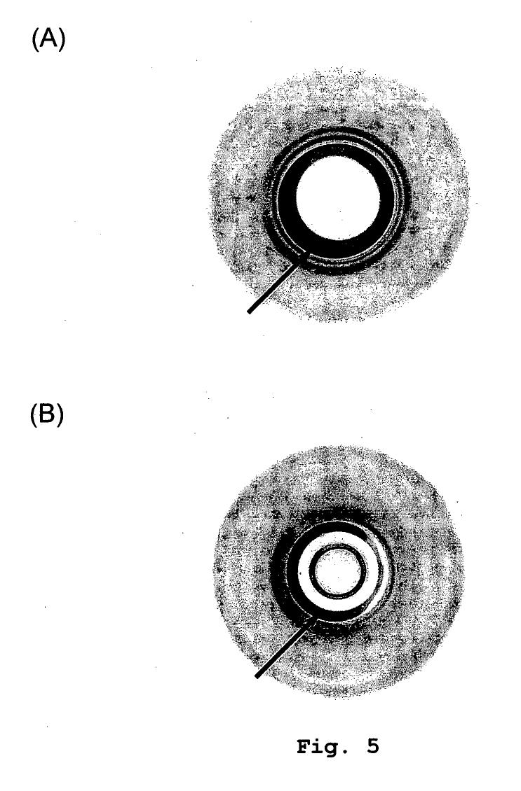

FIGS. 5A and 5B are photographs (view from above) of the rings, indicated by the arrow, that the red blood cells formed on the bottom of the microwells (inside of the peripheral chamber 4) after centrifugation;

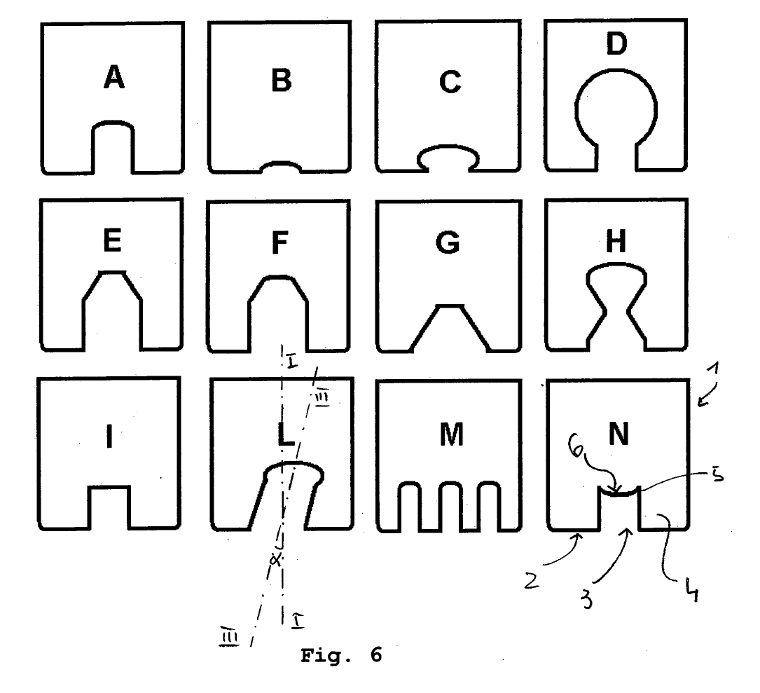

FIGS. 6A-6N represent—in section—twelve different embodiments of convex-bottom microwells according to the present invention;

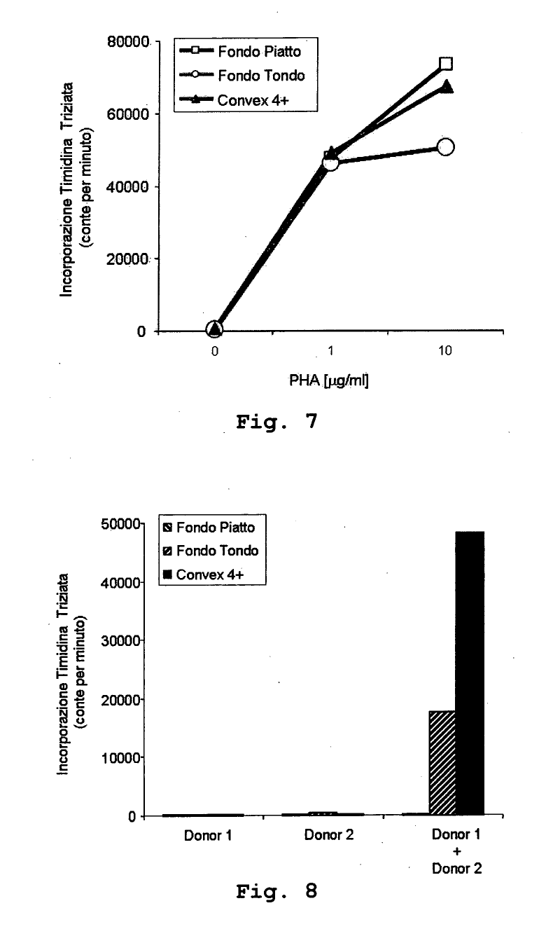

FIG. 7 represents a graph of the lymphocyte proliferation induced by increasing doses of phytohaemagglutinin. Comparison between convex-bottom microwells with inverted cylinder structure (denominated as Convex4+) and traditional plates with flat or round/concave bottoms;

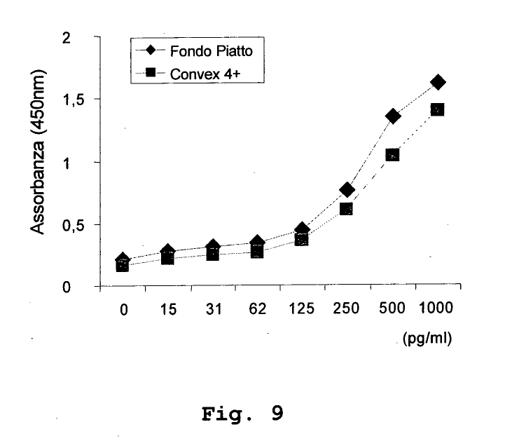

FIG. 8 represents a graph of the lymphocyte proliferation in a mixed allogenic lymphocyte reaction (MLR). Comparison between convex-bottom microwells with inverted cylinder structure (denominated as Convex4+) and traditional plates with flat and round/concave bottoms;

FIG. 9 represents a graph of a spectrophotometric reading of a standard calibration curve. Comparison between convex-bottom microwells with inverted cylinder structure (denominated as Convex4+) and traditional plates;

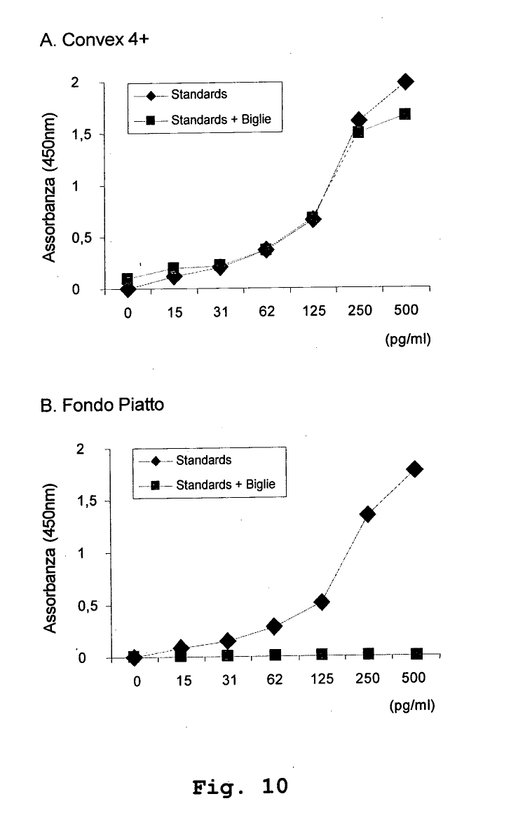

FIGS. 10A and 10B represent a spectrophotometric reading of a standard calibration curve in the presence or absence of a 35 □L film of magnetic microbeads. Comparison between convex-bottom microwells with inverted cylinder structure (denominated as Convex4+) (FIG. 10A) and traditional plates (FIG. 10B).

In a particular embodiment, the microwells according to the present invention include a protuberance on the bottom of the microwell so as to create a peripheral collecting chamber.

The employment of the above said microwells allows the collection of the interfering corpuscular particles by simply using the force of gravity, a centrifugation or magnetic field to reduce the operating time. In particular, the employment of the above said microwells allows the separation of the corpuscular parts in biological tests.

In addition, employment of the above said microwells allows the realisation of multiple-well plates, for example plates of 4, 6, 12, 24, 96 and 384 microwells. In addition, the technical solution described herein allow the realisation of microwells provided with, centrally to the convexity, a small collecting basin conveniently sized to collect a pre-fixed amount of corpuscular elements to analyse and then to discharge the excess, which will be collected in the peripheral chamber.

With reference to FIG. 1, a microwell 1 according to the solution described herein can be constituted of a cup or beaker of height “h” with a bottom 2 provided with a convex, that is, raised zone (3), of height “h1” inferior to “h”, preferably positioned centrally with respect to the longitudinal axis I-I of the microwell 1. The convex zone 3 can, in addition, have a surface area “a1” inferior to the surface area “a” of the bottom 2 of the microwell 1.

The convex zone 3 can assume different configurations as will be illustrated in greater detail below with reference to FIG. 6. It will be appreciated—with particular reference to the embodiment illustrated in FIG. 6N—that the term “convex” as used herein indicates the fact that zone 3 is generally raised with respect to the plane of the bottom 2 of the microwell 1. Therefore, such convex zone can have also concave or basin-like parts.

In a particular embodiment illustrated in FIG. 1, the convex zone 3 has a general inverted beaker or cylindrical structure (or simply an inverted cylinder structure) with a bottom 5 having, preferably a surface area “a1” inferior to the surface area “a” of the bottom 2 of the microwell 1, so as to create a peripheral chamber 4 having a general doughnut-like configuration. The peripheral chamber 4 preferably has a flat bottom.

Therefore, the microwell 1 according to the present invention following an operation of separation of the corpuscular phase C from the liquid phased L (FIG. 1B) allows the collection of the corpuscular phase C of the sample inside the peripheral chamber 4, allowing the luminous rays R emitted by a detecting instrument (not illustrated) to traverse the sample in correspondence to the convex zone 3 without being subject to reflections by the corpuscular phase C. In fact, the convex zone 3 is substantially transparent, whereby with the term transparent it is intended permeable to a radiation of analysis, also outside the visible field.

The operation of separation of the two phases does not involve the physical separation of the corpuscular phase C from the liquid phase L, but it allows the segregation of the corpuscular phase C in the peripheral chamber 4 of the microwell. Depending on the composition of the corpuscular phase (corpuscular elements in the blood, cells or beads possibly made of magnetic material) it is possible to have recourse to a separation by gravity, through centrifugation, or through application of a magnetic field.

In FIG. 6, different embodiments of a microwell according to the technical solution described herein are illustrated.

The FIGS. 6A and 6B represent microwells with convex bottoms having a convex zone 3 with so-called inverted U configuration (with respect to the longitudinal axis of the microwell 1) with heights h1 in their convex zones 3 that are different from each other.

FIGS. 6C and 6D are schematic representations of a microwell having a convex zone 3 with so-called omega and light bulb configurations.

FIGS. 6E, 6F and 6G represent a microwell having a convex zone 3 with an inverted V configuration, where, with this term it is intended a cone or pyramid configuration possibly truncated.

FIGS. 6H-6L are schematic representations of a microwell according to the present invention provided with convex zones 3 in the shape of: an hourglass, FIG. 6H; a cup or inverted cylinder, 6I; an inclined inverted cylinder (i.e. whose respective longitudinal axis III-III forms an angle α different from zero with respect to the longitudinal axis I-I of the microwell 1), 6L.

FIG. 6M illustrates a microwell having a plurality of convex zones 3 with an inverted cylinder general configuration.

A particular embodiment of a microwell according to the present invention is illustrated in FIG. 6N, where the convex zone 3 has a cup or inverted cylinder general structure whose respective bottom 5 presents a concave basin-like zone 6 preferably positioned centrally along the longitudinal axis of the microwell. The above said concave zone 6 is dimensioned to collect a certain quantity of corpuscular elements for analysis and then, it allows the discharging of the excess of such corpuscular elements which will be collected in the peripheral chamber 4.

The microwells according to the present technical solution allow a considerable simplification of all the procedures requiring the separation of interfering corpuscular elements present in the sample, thus reducing the execution times of the analyses. It does not call for higher production costs with respect to microplates formed with normal flat or concave microwells, neither in terms of materials employed nor in the construction of the machines. Some embodiments of the microwell according to the present invention illustrated in FIGS. 3 and 4 were realised employing four different 1-figure moulds. The bottom 2 of the microwell 1 presents a raised central curvature. The design phase is based on two presumptions: first, based on the consideration that the more marked the curvature, and therefore the more inclined the plane, the better the corpuscular elements flow during the centrifugation operation, therefore obtaining a more rapid and efficacious separation operation. The second presumption is based on the consideration that a greater curvature causes greater deviation of light; such negative characteristic has been evaluated experimentally.

Therefore, two different profiles of the convex zone 3 were designed and for each of them two subtypes having different central curvatures were designed. A profile definable as “horizontal C” with the meniscus towards the top, covers only one part of the bottom of the microwell, leaving a peripheral chamber 4 with a flat bottom as illustrated in FIG. 3.

A second type of profile, defined as “inverted U” also covers only one part of the bottom 2 of the microwell 1, leaving a peripheral chamber 4 with a flat bottom where the walls of the inverted U are perpendicular to the plane of the bottom 2 as illustrated in FIG. 4. The perimeter chambers 4 have the function of collecting the corpuscular elements after centrifugation.

In the designs illustrated in FIGS. 3 and 4, the indication T.A. (that is, transparent area) indicates that the convex zone 3 is constructed to be transparent to light. In that point, the moulds were chromed and mirror-polished.

The material used for the moulding of the microwells is transparent polystyrene (PS) conforming to the standards of the Food and Drug Administration, of the British Plastic Federation and to the prescriptions of the DGSIP n. 16 of Jul. 20, 1994 of the Ministero della Sanita'.

The material also conforms to anti-pollution laws, since the combustion residues—if this occurs in the presence of sufficient air and at an appropriate temperature—are composed of water and carbon dioxide.

The four microwells described above and represented in FIGS. 3 and 4 were then experimentally tested.

A test of the leukocyte vitality was set up, hypothesising the use of a specific dye capable of changing colour based on their viability. It is hypothesised that the dye does not act on erythrocytes, but that the erythrocytes themselves must be removed from the path of the optical ray in order to avoid interference. In this case, the erythrocytes are considered the disturbing corpuscular elements. Anti-coagulant (EDTA) treated blood was used at a leukocyte concentration equal to 5,000/μl, that is, 5,000,000/ml. An optimal concentration of 100,000 cells/ml was hypothesised. To bring the concentration of leukocytes to this cellularity, it is necessary to make a 1:50 dilution, that is 1 μl of blood is to be diluted with 49 μl of physiological solution, yielding a final volume of 50 μl.

In order to establish the total volume of sample to dispense in the respective microwells, the volume of the peripheral chamber 4 of the microwell 1 dedicated to collecting the corpuscular particles after centrifugation was kept in consideration. The different amounts of sample to dispense in each microwell were established hypothesising an elevated haematocrit (corpuscular part of the blood) value.

In order to evaluate the interfering effect of the red blood cells, a test in microwells with different characteristics was performed and the absorbance values were determined using physiological solution (0.9% NaCl) as the “blank”. 147 μl of physiological solution and 3 μl of blood were dispensed in the microwells illustrated in FIGS. 3 and 4. In addition, a test with a higher cellularity was performed by dispensing 135 μl of physiological solution and 15 μl of blood.

After having subjected the microwells to agitation, a pink-coloured suspension formed, interfering with the spectrophotometric reading.

Next, the microwells were subjected to two conditions of centrifugation: at 50 g for 5 minutes and 2,000 g for 10 minutes.

All tests with low blood concentration lead to the collection of the interfering cells in the peripheral chamber 4, leaving the aqueous solution clear; no microscopic traces of haemolysis were evident.

In FIG. 5, looking from above the microwell, we can observe the ring, indicated by the arrow, that the red blood cells formed on the bottom of the microwell (inside the peripheral chamber 4) after centrifugation. The different curvatures of the microwells are not a determinant for the result of the separation.

Depending on the different conditions of cellularity and centrifugation, the ring of red blood cells precipitated can be more or less complete, as is visible in FIG. 5.

Four types of microwells with different bottoms were tested: with different curve radius of the convex part and with different collecting chamber dimensions. A flat-bottom microwell was used as the control.

From the absorbance value obtained, it was possible to demonstrate that the interference of the red blood cells is eliminated following centrifugation: the high absorbance in the non-centrifuged solutions is reduced after this operation up to levels compatible with those of the controls with physiological solution.

Furthermore, it is possible to show the importance of the volume of the collecting space: an elevated number of red blood cells may not be completely caught in the peripheral chamber 4 of the microwell illustrated in FIG. 3A and therefore, recourse to a more suitable type of microwell such as that illustrated in FIGS. 4A and 4B is necessary. On the other hand, recourse to the latter type causes an increase in the basal absorbance value, probably due to the higher thickness of the plastic material traversed by the luminous ray. However, such characteristic does not invalidate the performance of the absorbance test since the “blank”, the “controls” and the samples are all subjected to the same conditions of analysis.

The results obtained showed that a profile made of a convex protuberance on the bottom of the microwell allows the creation of a peripheral chamber that serves to collect possible corpuscular particles interfering with the passage of an optical ray. Based on the results, such profile does not have a critical shape; therefore, while keeping the main characteristic, it can assume different shapes based on the specific applications. The microwells object of this patent can have different profiles studied to optimise the separation of the corpuscular elements with a single centrifugation.

### EXAMPLES

In the following, some examples will be given directed at evaluation of the use of convex-bottom microwells according to the present invention. The analyses were performed employing a 300 μl microwell in 96-well plates. The single microwells were mounted on 8-well ELISA racks.

### Example

### Cell Culture

a) Use of Inverted Cylinder Convex-Bottom Wells for the Evaluation of Lymphocyte Response to Mitogens

It was evaluated whether the prototype inverted cylinder convex-bottom microwell (single well) denominated Convex4+ is suitable to allow lymphocyte growth, comparing its use with that of flat and round bottom (also called concave or U-bottom) traditional 96-well culturing plates.

Peripheral blood lymphocytes purified on a Ficoll density gradient were seeded in triplicate with a cell number of 100,000 for each well in a final volume of 200 μl of culturing media, (RPMI+10% foetal bovine serum) and stimulated with increasing doses of Phytohemagglutinin (PHA). After 72 hours in culture, the cellular response in terms of proliferation was measured by detecting the incorporation of Tritiated Thymidine (0.5 μCi/well) during the last 6 hours of culturing, using a beta radiation counter (the analysis was performed on cells collected with a semi-automatic cell harvester). As is shown in FIG. 7, the inverted cylinder convex microwell allows proliferation levels to be obtained that are completely comparable to those obtained with traditional wells and therefore, it is suitable for use in cell culture.

b) Use of the Inverted Cylinder Convex Microwell to Evaluate Mixed Lymphocyte Reactions (MLR)

The use of inverted cylinder convex microwells denominated Convex4+ was evaluated in a bi-directional allogenic mixed lymphocyte reaction (MLR), in which lymphocyte activation depends strictly on cell contact between the lymphocytes of two donors and from the reciprocal recognition as non-self. We compared the efficiency of Convex4+ inverted cylinder convex microwells with that of traditional plates with round or flat-bottom wells (this type of experiment is generally performed on plates with round-bottom wells since they favour intercellular contact). Lymphocytes of each donor (1 and 2) were seeded alone (1 or 2) or together (1+2) in the wells at 50,000 cells/well in a final volume of 100 μl, without adding other stimuli. Cellular response in terms of proliferation was evaluated as above by detecting the incorporation of [³H]-TdR in the last six hours of the five days of culture.

As is shown in FIG. 8, these experiments demonstrated that the Convex4+ microwell favours the lymphocyte response in this type of assay, yielding results notably superior with respect to flat-bottom wells (particularly inefficacious in these assays), but showing a certain level of advantage also compared to the round-bottom ones which represent the standard for these tests.

This first series of experiments allows us to conclude that the use of inverted cylinder convex microwells in cell culture is a valid alternative to traditional plates.

### Example 2

### ELISA Test

a) Use of Inverted Cylinder Convex Microwells for ELISA Readings

We evaluated the possible use of inverted cylinder convex microwells denominated Convex4+ in an ELISA (enzyme-linked immunosorbent assay). The coloured reaction product of a calibration curve of an ELISA reaction was loaded in the inverted cylinder convex microwells Convex4+ and in traditional flat-bottom ELISA plates (concave-bottom plates cannot be used in these types of assays) in the amount of 100 μl per microwell and then we compared the efficiency of the spectrophotometer reading.

As can be observed in FIG. 9, the relative optical density of the different points of a calibration curve detected using the inverted cylinder convex microwells Convex4+ is completely comparable with that obtained with traditional plates.

This result demonstrates that the inverted cylinder convex microwells are suitable for use in an ELISA reader.

b) Use of Inverted Cylinder Convex Microwells for ELISA Readings in the Presence of Opaque Corpuscular Material

We evaluated whether the inverted cylinder convex microwell denominated Convex4+ allows the reading of an ELISA standard calibration curve, in the presence of magnetic microbeads deposited in the wells and trapped on the bottom of the well by a magnetic pad. We used a volume of 30 μl of magnetic microbeads, which completely covered the bottom of a flat-bottom well, but that remained confined in the peripheral chamber 4 of the bottom 2 of the Convex4+ microwell leaving the raised transparent central zone (convex zone 3) of the microwell free.

A standard calibration curve for ELISA was seeded in the wells and read with a spectrophotometer in the presence or absence of the beads on plates with flat-bottom microwells or Convex4+ convex microwells.

In FIG. 10A it can be seen that the Convex4+ microwells allowed the reading of the standard curve also in the presence of microbeads; FIG. 10B shows the result obtained in plates with flat-bottom microwells, in which the presence of beads did not allow the spectrophotometer to give numeric values (in the figure they are shown with the value zero for simplicity).

This result demonstrates that the inverted cylinder convex microwells are completely suitable in allowing an ELISA reading in the presence of opaque particles, while this is not possible using a traditional ELISA plate.

Naturally, the particulars of realisation and the embodiments could be widely varied with respect to what is described and illustrated, without going outside of the field of protection of the present invention as defined in the attached claims.

1. A microwell (1) characterised in that it includes a protuberance on the bottom (2) of the microwell (1) so as to create a peripheral collecting chamber (4).

2. A microwell according to claim 1, in which said microwell includes a beaker-like structure and said protuberance is constituted by at least one convex zone (3).

3. A microwell according to claim 2, in which said microwell (1) has a height “h” and a bottom (2) of surface area “a” and in which said at least one convex zone (3) has a height “h1” inferior to the height “h” of said microwell (1).

4. A microwell according to claim 3, in which said at least one convex zone (3) has a surface area “a1” inferior to the surface area “a” of said bottom (2) of said microwell (1).

5. A microwell according to claim 1, in which said microwell (1) has in addition at least one peripheral chamber (4) positioned around said at least one convex zone (3), said peripheral chamber (4) having a doughnut-like configuration.

6. A microwell according to claim 2, in which said at least one convex zone (3) is centrally positioned with respect to the longitudinal axis I-I of the microwell (1).

7. A microwell according to claim 2, in which said at least one convex zone (3) has an inverted cylinder structure.

8. A microwell according to claim 2, in which said at least one convex zone (3) has an inverted U structure.

9. A microwell according to claim 2, in which said at least one convex zone (3) has an omega or light bulb structure.

10. A microwell according to claim 2, in which said at least one convex zone (3) has a cone or pyramid structure optionally truncated.

11. A microwell according to claim 2, in which said at least one convex zone (3) has an hourglass structure.

12. A microwell according to claim 2, in which said at least one convex zone (3) has an inclined inverted cylinder structure.

13. A microwell according to claim 1, in which said at least one convex zone (3) has a bottom (5) provided with a basin-like concave zone (6).

14. A microwell according to claim 13, in which said concave zone (6) is centrally positioned along the longitudinal axis I-I of the microwell (1).

15. A microwell according to claim 1, in which said at least one convex zone (3) is transparent.

16. A microwell according to claim 1, in which said microwell is made of transparent polystyrene.

17. A microplate having a plurality of microwells according to claim 1.

18. The use of a microwell according to claim 1 for performing a microbiological and/or enzymological test on a biological sample.

19. The use according to claim 18 in which said biological sample is selected among blood, plasma, serum, cell culture.

20. The realization and use according to claim 1 of a microwell collecting interfering corpuscular particles simply by using the force of gravity, a centrifugation or a magnetic field to reduce operating times.

21. The realization according to claim 1 of multiple-well plates.

22. The realization according to claim 21 of plates with 4, 6, 12, 24, 96 and 184 microwells.

23. The realization and use according to claim 1 of microwells or plates for the separation of the corpuscular parts in biological tests.

24. The realization and use according to claim 1, of microwells having a small collecting basin centrally to the convexity, suitably dimensioned to collect a pre-fixed portion of corpuscular elements for analysis and then to discharge the excess, which will be collected in the peripheral chamber.
